(19) United States
(12) Patent Application Publication
(10) Pub. No.: US 2022/0207221 A1
(43) Pub. Date: Jun. 30, 2022

(54) METHOD AND APPARATUS FOR SIMULATING PHYSIOLOGICAL DYNAMIC BLOOD FLOW, COMPUTER DEVICE AND STORAGE MEDIUM

(71) Applicant: Shenzhen Technology University, Shenzhen (CN)

(72) Inventors: Zhengwei MA, Shenzhen (CN); Jiaxing CHEN, Shenzhen (CN); Changren QIU, Shenzhen (CN)

(73) Assignee: Shenzhen Technology University, Shenzhen (CN)

(21) Appl. No.: 17/459,766

(22) Filed: Aug. 27, 2021

(30) Foreign Application Priority Data
Dec. 29, 2020 (CN) 202011589928.X

(51) Int. Cl. G06F 30/28; G06F 30/23; G16H 50/50

(52) U.S. Cl. CPC G06F 30/28; G06F 2113/08; G16H 50/50; G06F 30/23

(57) ABSTRACT

The present disclosure provides a method and apparatus for simulating a physiological dynamic blood flow, a computer device and a storage medium. The method for simulating a physiological dynamic blood flow includes the following steps: acquiring related parameters of a cardio-aortic system in a complete cardiac cycle under a normal physiological state, and acquiring morphological and motion imaging data of the cardio-aortic system in the complete cardiac cycle; constructing a circulation dynamic loading function for cyclic dynamic contraction of a left ventricle; constructing a function between a left ventricular (LV) dynamic blood pressure and an LV dynamic volume; constructing a dynamic arterial blood flow volume function; constructing a cyclic opening and closing activation function of an aortic valve; and constructing a physiological dynamic blood flow model, and simulating the physiological dynamic blood flow according to the physiological dynamic blood flow model.

### CROSS REFERENCE TO RELATED APPLICATION

This patent application claims the benefit and priority of Chinese Patent Application No. 202011589928.X filed on Dec. 29, 2020, the disclosure of which is incorporated by reference herein in its entirety as part of the present application.

### TECHNICAL FIELD

The present disclosure relates to the field of simulating physiological dynamic blood flow, and in particular, to a method and apparatus for simulating a physiological dynamic blood flow, a computer device and a storage medium.

### BACKGROUND

Cardiovascular trauma and related diseases are now one of the most primary causes of death globally. Particularly in vehicle collisions, traumatic rupture of the aorta (TRA) accounts for more than 20% of all accidental deaths, and is the second major cause of death for passengers next to the craniocerebral injury. The TRA is an acute severe injury. More than 85% of people afflicted with the aortic injury will die at the scene of accident, and more than 50% of survivals will die within 24 h if not effectively treated. However, the aortic injury is generally not easy to be detected when the emergency rescue is given to the injured people. Even in most cases, the passenger does not suffer it at the accident site but often shows the TRA at an uncertain time thereafter and dies soon. The TRA research has always been an important focus in the fields of medical, epidemiology of accidental injuries, safety technologies and so on. On one hand, obtaining a traumatic mechanism and an injury tolerance of the TRA in the vehicle collisions can offer effective guidance on safety designs of vehicles and roads to reduce the risk of TRA in the collisions; and on the other hand, grasping accident characteristics and prediction criteria for the occurrence of the TRA can be helpful for surgeons to make a more prompt and accurate diagnosis on the people afflicted with the aortic injury to lower the risk of misdiagnosis.

In recent years, with continuous improvements of computer technologies and finite element methods, construction of digital models having real anatomical structures and biomechanical characteristics has become a new method for the TRA research. There are increasingly more researchers to research the physiological structure, functional characteristic, injury mechanism, tolerance limit and other issues of a cardio-aortic system by constructing different types of virtual digital models. The aortic blood flow in a physiological circulation of human bodies is dynamically changing, and the difference between high and low blood pressures may be up to more than one third of a peak blood pressure. It is found in the earlier work that an abrupt change in the blood pressure under the combined action of a collision load to the chest of the passenger and a physiological dynamic blood flow of the aorta is a critical factor for inducing the TRA in vehicle collision. However, due to the complexity and research difficulty of the cardio-aortic system, most of the existing models have ignored the impact originating from the physiological dynamic blood flow of the body aortic system.

### SUMMARY

An object of the present disclosure is to provide a method and apparatus for simulating a physiological dynamic blood flow, a computer device and a storage medium, to overcome the defects of the prior art.

To achieve the above object, the present disclosure employs the following technical solution.

The present disclosure provides a method for simulating a physiological dynamic blood flow, including the following steps:

acquiring related parameters of a cardio-aortic system in a complete cardiac cycle under a normal physiological state, and acquiring morphological and motion imaging data of the cardio-aortic system in the complete cardiac cycle;

constructing a circulation dynamic loading function for cyclic dynamic contraction of a left ventricle according to the morphological and motion imaging data of the cardio-aortic system in the complete cardiac cycle;

constructing a function between an LV dynamic blood pressure and an LV dynamic volume according to the morphological and motion imaging data of the cardio-aortic system in the complete cardiac cycle;

constructing a dynamic arterial blood flow volume function according to the related parameters of the cardio-aortic system in the complete cardiac cycle under the normal physiological state;

constructing a cyclic opening and closing activation function of an aortic valve based on the morphological and motion imaging data of the cardio-aortic system in the complete cardiac cycle; and

constructing a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulating the physiological dynamic blood flow according to the physiological dynamic blood flow model.

As a further technical solution, the step of acquiring related parameters of a cardio-aortic system in a complete cardiac cycle under a normal physiological state includes:

acquiring an arterial blood pressure, a venous blood pressure, an arterial compliance and a peripheral impedance of the cardio-aortic system in the complete cardiac cycle under the normal physiological state.

As a further technical solution, the step of constructing a circulation dynamic loading function for cyclic dynamic contraction of a left ventricle according to the morphological and motion imaging data of the cardio-aortic system in the complete cardiac cycle includes:

extracting a medical image of the cardio-aortic system at a maximum end diastolic volume and a minimum end systolic volume of the left ventricle from morphological and motion imaging data of the cardio-aortic system in one complete cardiac cycle; and

performing modeling and mapping according to the medical image of the cardio-aortic system to obtain the circulation dynamic loading function for the cyclic dynamic contraction of the left ventricle.

As a further technical solution, the step of performing modeling and mapping according to the medical image of the cardio-aortic system to obtain the circulation dynamic loading function for the cyclic dynamic contraction of the left ventricle includes:

constructing a maximum end diastolic volume model and a minimum end systolic volume model respectively according to the medical image of the cardio-aortic system through image extracting, geometric modeling and finite element modeling;

constructing a grid unit node mapping between the maximum end diastolic volume model and the minimum end systolic volume model to determine a point-to-point corresponding relation between the grid unit nodes;

establishing an LV volume change function V_{LV}(t) according to the morphological and motion imaging data of the cardio-aortic system in the complete cardiac cycle; and

obtaining a motion displacement function Dᵢ(t) on the basis of the mapping in combination with the LV volume change function V_{LV}(t), and taking the motion displacement function Dᵢ(t) as the circulation dynamic loading function for the cyclic dynamic contraction of the left ventricle.

As a further technical solution, the step of constructing a function between an LV dynamic blood pressure and an LV dynamic volume according to the morphological and motion imaging data of the cardio-aortic system in the complete cardiac cycle includes:

constructing the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)V_{LV}(t)−V_{(LVmin)}+E_{P}(V_{LV}(t)−V_{LVmax}), where P_{LV}(t) is the LV dynamic blood pressure, V_{LV}(t) is the LV dynamic volume, V_{Lvmax} is the maximum end diastolic volume, V_{LVmin} is the minimum end systolic volume, E_{A}(t) is a time-varying elasticity coefficient for LV myocardial active contraction, and E_{A}(t) is a passive elasticity coefficient.

As a further technical solution, the step of constructing a dynamic arterial blood flow volume function according to the related parameters of the cardio-aortic system in the complete cardiac cycle under the normal physiological state includes:

constructing the dynamic arterial blood flow volume function according to the arterial blood pressure, the venous blood pressure, the arterial compliance and the peripheral impedance by employing

$$Q ( t ) = C \frac{dP ( t )}{d ι} + \frac{P ( t ) - {P}_{V}}{{R}_{P}} ,$$

where Q(t) is an arterial blood flow volume, P(t) is the arterial blood pressure, P_{V} is the venous blood pressure, C is the arterial compliance, and R_{P} is the peripheral impedance.

As a further technical solution, the step of constructing a cyclic opening and closing activation function of an aortic valve based on the morphological and motion imaging data of the cardio-aortic system in the complete cardiac cycle includes:

defining, based on the maximum end diastolic volume model and the minimum end systolic volume model, a region of the cardio-aortic system as an Euler fluid grid filled with two materials, with an LV wall and an aortic wall as dynamic limits, and the inside is automatically defined as a blood fluid, and the outside is automatically defined as an air fluid; and

controlling a contact relation between the region-defined aortic valve and blood with 0-1 activated states of a preset function A(t), and reversely controlling a contact relation between the region-defined LV wall and the blood with 0-1 activated states of a preset function |A(t)−1|through an ALE algorithm, thereby constructing the cyclic opening and closing activation function of the aortic valve.

The present disclosure further provides an apparatus for simulating a physiological dynamic blood flow, including:

an acquisition unit, configured to acquire related parameters of a cardio-aortic system in a complete cardiac cycle under a normal physiological state, and morphological and motion imaging data of the cardio-aortic system in the complete cardiac cycle;

a first construction unit, configured to construct a circulation dynamic loading function for cyclic dynamic contraction of a left ventricle according to the morphological and motion imaging data of the cardio-aortic system in the complete cardiac cycle;

a second construction unit, configured to construct a function between an LV dynamic blood pressure and an LV dynamic volume according to the morphological and motion imaging data of the cardio-aortic system in the complete cardiac cycle;

a third construction unit, configured to construct a dynamic arterial blood flow volume function according to the related parameters of the cardio-aortic system in the complete cardiac cycle under the normal physiological state;

a fourth construction unit, configured to construct a cyclic opening and closing activation function of an aortic valve based on the morphological and motion imaging data of the cardio-aortic system in the complete cardiac cycle; and

a model generation unit, configured to construct a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulate the physiological dynamic blood flow according to the physiological dynamic blood flow mode.

The present disclosure further provides a computer device, including a memory and a processor, wherein a computer program is stored on the memory, and when executed by the processor, the computer program implements the method as described above for simulating a physiological dynamic blood flow.

The present disclosure further provides a storage medium, storing a computer program, wherein when executed by a processor, the computer program implements the method as described above for simulating a physiological dynamic blood flow.

Compared with the prior art, the beneficial effects of the present disclosure are summarized as follows. By constructing a circulation dynamic loading function for cyclic dynamic contraction of a left ventricle, a function between an LV dynamic blood pressure and an LV dynamic volume, a dynamic arterial blood flow volume function and a cyclic opening and closing activation function of an aortic valve, and constructing a physiological dynamic blood flow model based on the above functions, the method for simulating the physiological dynamic blood flow provided by the present disclosure implements simulation on dynamic physiological characteristics of the cardio-aortic system in the complete cardiac cycle, and is used in the fields of life science, medicines, biomechanics, rehabilitation engineering and safety technologies to research the physiological characteristics, functional injuries, injury mechanisms and the like and an injury diagnosis and treatment of the cardio-aortic system under a virtual state, and effectively improves the level of accuracy of modeling and research on functions and injuries of the cardio-aortic system.

The present disclosure is further described below in combination with the accompanying drawings and specific embodiments.

### BRIEF DESCRIPTION OF THE DRAWINGS

In order to describe the technical solutions in the embodiments of the present disclosure more clearly, the accompanying drawings required to describe the embodiments are briefly described below. Apparently, the accompanying drawings described below are only some embodiments of the present disclosure. Those of ordinary skill in the art may further obtain other accompanying drawings based on these accompanying drawings without creative efforts.

FIG. 1 is a schematic view of an application scenario of a method for simulating a physiological dynamic blood flow provided by an embodiment of the present disclosure.

FIG. 2 is a flow chart of a method for simulating a physiological dynamic blood flow provided by an embodiment of the present disclosure.

FIG. 3 is a sub flow chart of a method for simulating a physiological dynamic blood flow provided by an embodiment of the present disclosure.

FIG. 4 is a sub flow chart of a method for simulating a physiological dynamic blood flow provided by an embodiment of the present disclosure.

FIG. 5 is a sub flow chart of a method for simulating a physiological dynamic blood flow provided by an embodiment of the present disclosure.

FIG. 6 is a schematic block diagram of an apparatus for simulating a physiological dynamic blood flow provided by an embodiment of the present disclosure.

FIG. 7 is a schematic block diagram of an acquisition unit of an apparatus for simulating a physiological dynamic blood flow provided by an embodiment of the present disclosure.

FIG. 8 is a schematic block diagram of a first construction unit of an apparatus for simulating a physiological dynamic blood flow provided by an embodiment of the present disclosure.

FIG. 9 is a schematic block diagram of a modeling and mapping module of an apparatus for simulating a physiological dynamic blood flow provided by an embodiment of the present disclosure.

FIG. 10 is a schematic block diagram of a second construction unit of an apparatus for simulating a physiological dynamic blood flow provided by an embodiment of the present disclosure.

FIG. 11 is a schematic block diagram of a third construction unit of an apparatus for simulating a physiological dynamic blood flow provided by an embodiment of the present disclosure.

FIG. 12 is a schematic block diagram of a fourth construction unit of an apparatus for simulating a physiological dynamic blood flow provided by an embodiment of the present disclosure.

FIG. 13 is a schematic block diagram of a computer device provided by an embodiment of the present disclosure.

### DETAILED DESCRIPTION OF THE EMBODIMENTS

The technical solutions in the embodiments of the present disclosure will be described below clearly and completely in combination with the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

It should be understood that when used in this specification and the appended claims, the terms “comprise” and “include” indicate the presence of described features, integers, steps, operations, elements and/or components, but do not exclude the presence or addition of one or more of other features, integers, steps, operations, elements, components, and/or sets thereof

It should also be understood that the terms used in the specification of the present disclosure are for the purpose of describing specific embodiments only and are not intended to limit the present disclosure. As used in the specification of the present disclosure and the appended claims, the singular forms “a”, “an” and “the” are intended to include the plural forms unless the context clearly indicates otherwise.

It should also be further understood that the term “and/or” used in the specification of the present disclosure and the appended claims refers to one or any or all possible combinations of multiple associated items that are listed, and includes these combinations.

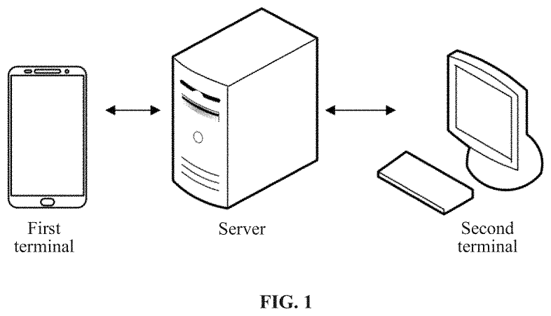

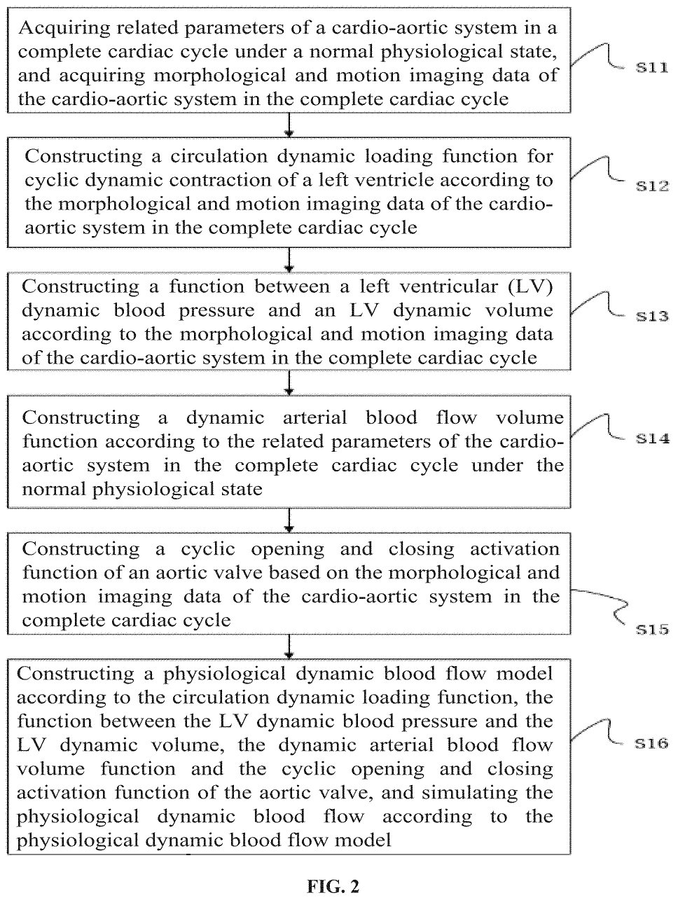

Referring to FIG. 1 and FIG. 2, FIG. 1 is a schematic view of an application scenario of a method for simulating a physiological dynamic blood flow provided by an embodiment of the present disclosure, and FIG. 2 is a flow chart of a method for simulating a physiological dynamic blood flow provided by an embodiment of the present disclosure. The method for simulating the physiological dynamic blood flow is applicable to a server. Data interaction between the server and a terminal is conducted, and the terminal is used to capture to-be-recognized image data and transmit the to-be-recognized image data to the server. A text recognition module in the server performs text recognition on the to-be-recognized image data, and aligns a recognition result to obtain a real character sequence, i.e., text information. The text information may be transmitted to the terminal or the text information may be used to control the terminal to make a corresponding response.

FIG. 2 is a flow chart of a method for simulating a physiological dynamic blood flow provided by an embodiment of the present disclosure. As shown in FIG. 2, the method may include the following steps S11 to S16.

S11, acquiring related parameters of a cardio-aortic system in a complete cardiac cycle under a normal physiological state, and acquiring morphological and motion imaging data of the cardio-aortic system in the complete cardiac cycle.

In the embodiment, the related parameters of the cardio-aortic system in the complete cardiac cycle under the normal physiological state, and the morphological and motion imaging data of the cardio-aortic system in the complete cardiac cycle are acquired to facilitate the subsequent processing and modeling according to these related parameters and imaging data.

S12, constructing a circulation dynamic loading function for cyclic dynamic contraction of a left ventricle according to the morphological and motion imaging data of the cardio-aortic system in the complete cardiac cycle.

S13, constructing a function between an LV dynamic blood pressure and an LV dynamic volume according to the morphological and motion imaging data of the cardio-aortic system in the complete cardiac cycle.

S14, constructing a dynamic arterial blood flow volume function according to the related parameters of the cardio-aortic system in the complete cardiac cycle under the normal physiological state.

S15, constructing a cyclic opening and closing activation function of an aortic valve based on the morphological and motion imaging data of the cardio-aortic system in the complete cardiac cycle.

S16, constructing a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulating the physiological dynamic blood flow according to the physiological dynamic blood flow model.

In the embodiment, by constructing the circulation dynamic loading function for cyclic dynamic contraction of a left ventricle, the function between an LV dynamic blood pressure and an LV dynamic volume, the dynamic arterial blood flow volume function and the cyclic opening and closing activation function of an aortic valve, and constructing the physiological dynamic blood flow model based on the above functions, the method implements simulation on dynamic physiological characteristics of the cardio-aortic system in the complete cardiac cycle, and is used in the fields of life science, medicines, biomechanics, rehabilitation engineering and safety technologies to research the physiological characteristics, functional injuries, injury mechanisms and the like and an injury diagnosis and treatment of the cardio-aortic system under a virtual state, and effectively improves the level of accuracy of modeling and research on functions and injuries of the cardio-aortic system.

In an embodiment, step S11 may include step S111.

S111, acquiring an arterial blood pressure, a venous blood pressure, an arterial compliance and a peripheral impedance of the cardio-aortic system in the complete cardiac cycle under the normal physiological state.

In the embodiment, these parameters such as the arterial blood pressure, the venous blood pressure, the arterial compliance and the peripheral impedance are acquired to facilitate the subsequent calculation to obtain the dynamic arterial blood flow volume.

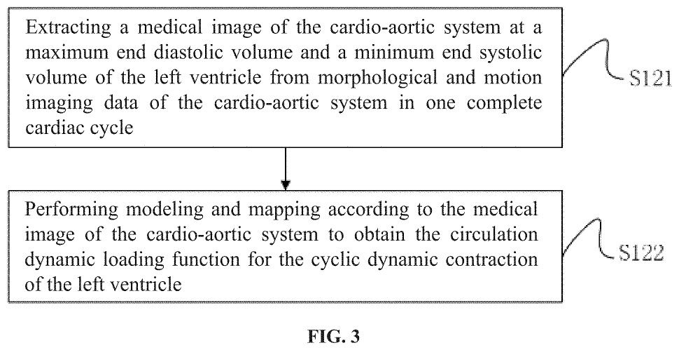

In an embodiment, as shown in FIG. 3, step S12 may include steps S121 to S122.

S121, extracting a medical image of the cardio-aortic system at a maximum end diastolic volume and a minimum end systolic volume of the left ventricle from morphological and motion imaging data of the cardio-aortic system in one complete cardiac cycle.

S122, performing modeling and mapping according to the medical image of the cardio-aortic system to obtain the circulation dynamic loading function for the cyclic dynamic contraction of the left ventricle.

In the embodiment, the physiological pulsation function for the left ventricle of the heart is implemented through model mapping from the maximum end diastolic volume to the minimum end systolic volume of the left ventricle of the heart.

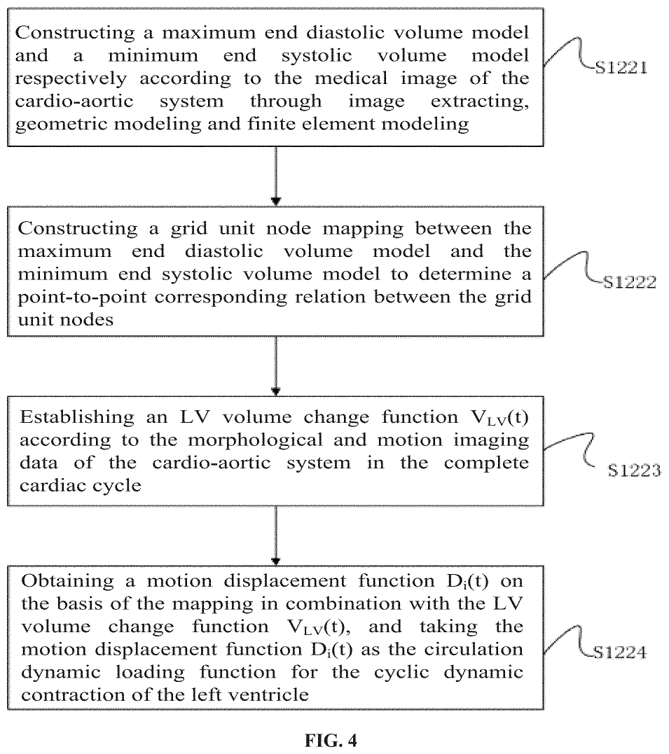

In an embodiment, as shown in FIG. 4, step S122 may include steps S1221 to S1224.

S1221, constructing a maximum end diastolic volume model and a minimum end systolic volume model respectively according to the medical image of the cardio-aortic system through image extracting, geometric modeling and finite element modeling.

S1222, constructing a grid unit node mapping between the maximum end diastolic volume model and the minimum end systolic volume model to determine a point-to-point corresponding relation between the grid unit nodes.

S1223, establishing an LV volume change function V_{LV}(t) according to the morphological and motion imaging data of the cardio-aortic system in the complete cardiac cycle.

S1224, obtaining a motion displacement function Dᵢ(t) on the basis of the mapping in combination with the LV volume change function V_{LV}(t), and taking the motion displacement function Dᵢ(t) as the circulation dynamic loading function for the cyclic dynamic contraction of the left ventricle.

In the embodiment, the physiological pulsation function for the left ventricle of the heart is implemented through model mapping from the maximum end diastolic volume to the minimum end systolic volume of the left ventricle of the heart based on the medical image of the cardio-aortic system and the finite element method.

In an embodiment, step S13 may include step S131.

S131, constructing the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)(V_{LV}(t)−V_{LVmin})+E_{P}(V_{LV}(t)−V_{LVmax}), where P_{LV}(t) is the LV dynamic blood pressure, V_{LV}(t) is the LV dynamic volume, V_{LVmax} is the maximum end diastolic volume, V_{LVmin} is the minimum end systolic volume, E_{A}(t) is a time-varying elasticity coefficient for LV myocardial active contraction, and E_{P} is a passive elasticity coefficient.

In the embodiment, the simulation of aspects from pulsation of the heart to the dynamic blood pressure is implemented by constructing the dynamic relation function between the volume and the pressure of the left ventricle, which can effectively improve the level of accuracy of modeling and research on functions and injuries of the cardio-aortic system.

In an embodiment, step S14 may include step S141.

S141, constructing the dynamic arterial blood flow volume function according to the arterial blood pressure, the venous blood pressure, the arterial compliance and the peripheral impedance by employing

$$Q ( t ) = C \frac{dP ( t )}{d ι} + \frac{P ( t ) - {P}_{V}}{{R}_{P}} ,$$

where Q(t) is an arterial blood flow volume, P(t) is the arterial blood pressure, P_{V} is the venous blood pressure, C is the arterial compliance, and R_{P} is the peripheral impedance.

In the embodiment, the simulation of aspects from pulsation of the heart to the dynamic blood pressure is implemented by constructing the Dynamic arterial blood flow function, which can effectively improve the level of accuracy of modeling and research on functions and injuries of the cardio-aortic system.

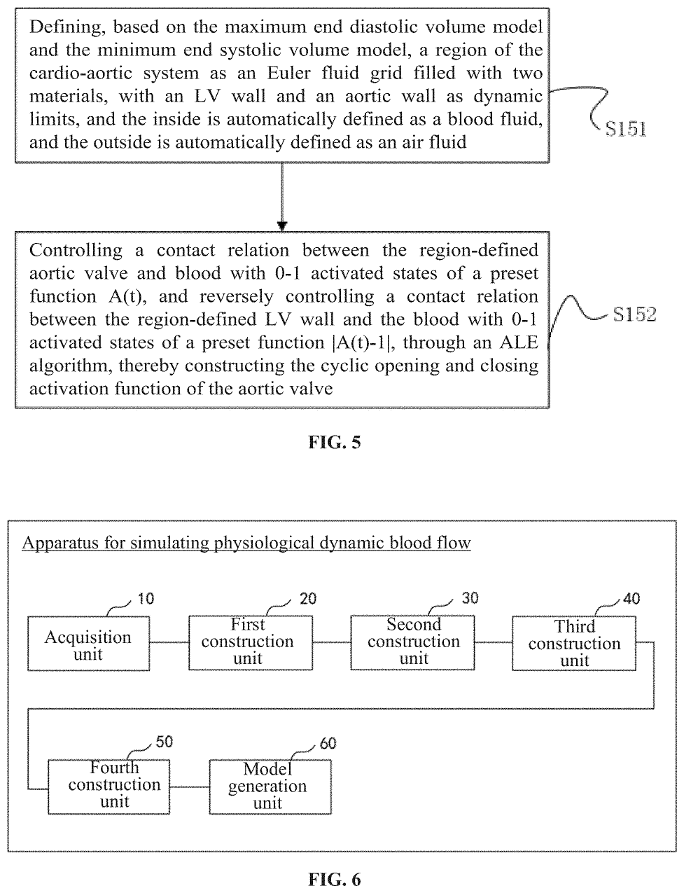

In an embodiment, as shown in FIG. 5, step S15 may include steps S151 and S152.

S151, defining, based on the maximum end diastolic volume model and the minimum end systolic volume model, a region of the cardio-aortic system as an Euler fluid grid filled with two materials, with an LV wall and an aortic wall as dynamic limits, and the inside is automatically defined as a blood fluid, and the outside is automatically defined as an air fluid.

S152, controlling a contact relation between the region-defined aortic valve and blood with 0-1 activated states of a preset function A(t), and reversely controlling a contact relation between the region-defined LV wall and the blood with 0-1 activated states of a preset function |A(t)−1|, through an ALE algorithm, thereby constructing the cyclic opening and closing activation function of the aortic valve.

In the embodiment, the contact relation between the region-defined aortic valve and the blood is controlled with the 0-1 activated states of the preset function A(t) (activated in a diastolic phase to avoid blood reflux of the aorta; and deactivated in a systolic phase to ensure blood pumping of the ventricle), and the contact relation between the region-defined LV wall and the blood is reversely controlled with the 0-1 activated states of the preset function |A(t)−1| (activated in the systolic phase to ensure blood pumping of the ventricle; and deactivated in the diastolic phase to ensure blood filling of the ventricle), to construct the cyclic opening and closing activation function of the aortic valve, and ensure that dynamic functional simulation of the constructed model is close to a physiological cyclic change of a real cardio-aortic system. The method implements simulation on dynamic physiological characteristics of the cardio-aortic system in the complete cardiac cycle, and is used in the fields of life science, medicines, biomechanics, rehabilitation engineering and safety technologies to research the physiological characteristics, functional injuries, injury mechanisms and the like and an injury diagnosis and treatment of the cardio-aortic system under a virtual state.

FIG. 6 is a schematic block diagram of an apparatus for simulating a physiological dynamic blood flow provided by an embodiment of the present disclosure. As shown in FIG. 6, corresponding to the method for simulating the physiological dynamic blood flow, the present disclosure further provides an apparatus for simulating a physiological dynamic blood flow. The apparatus for simulating a physiological dynamic blood flow includes units for executing the method for simulating the physiological dynamic blood flow as described above. The apparatus may be configured in terminals such as a desktop computer, a tablet computer and a laptop computer. Specifically, referring to FIG. 6, the apparatus for simulating a physiological dynamic blood flow include: an acquisition unit 10, a first construction unit 20, a second construction unit 30, a third construction unit 40, a fourth construction unit 50 and a model generation unit 60.

The acquisition unit 10 is configured to acquire related parameters of a cardio-aortic system in a complete cardiac cycle under a normal physiological state, and morphological and motion imaging data of the cardio-aortic system in the complete cardiac cycle.

In the embodiment, the related parameters of the cardio-aortic system in the complete cardiac cycle under the normal physiological state, and the morphological and motion imaging data of the cardio-aortic system in the complete cardiac cycle are acquired to facilitate the subsequent processing and modeling according to these related parameters and imaging data.

The first construction unit 20 is configured to construct a circulation dynamic loading function for cyclic dynamic contraction of a left ventricle according to the morphological and motion imaging data of the cardio-aortic system in the complete cardiac cycle.

The second construction unit 30 is configured to construct a function between an LV dynamic blood pressure and an LV dynamic volume according to the morphological and motion imaging data of the cardio-aortic system in the complete cardiac cycle.

The third construction unit 40 is configured to construct a dynamic arterial blood flow volume function according to the related parameters of the cardio-aortic system in the complete cardiac cycle under the normal physiological state.

The fourth construction unit 50 is configured to construct a cyclic opening and closing activation function of an aortic valve based on the morphological and motion imaging data of the cardio-aortic system in the complete cardiac cycle.

The model generation unit 60 is configured to construct a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulate the physiological dynamic blood flow according to the physiological dynamic blood flow model.

In the embodiment, by constructing the circulation dynamic loading function for cyclic dynamic contraction of a left ventricle, the function between an LV dynamic blood pressure and an LV dynamic volume, the dynamic arterial blood flow volume function and the cyclic opening and closing activation function of an aortic valve, and constructing the physiological dynamic blood flow model based on the above functions, the apparatus implements simulation on dynamic physiological characteristics of the cardio-aortic system in the complete cardiac cycle, and is used in the fields of life science, medicines, biomechanics, rehabilitation engineering and safety technologies to research the physiological characteristics, functional injuries, injury mechanisms and the like and an injury diagnosis and treatment of the cardio-aortic system under a virtual state, and effectively improves the level of accuracy of modeling and research on functions and injuries of the cardio-aortic system.

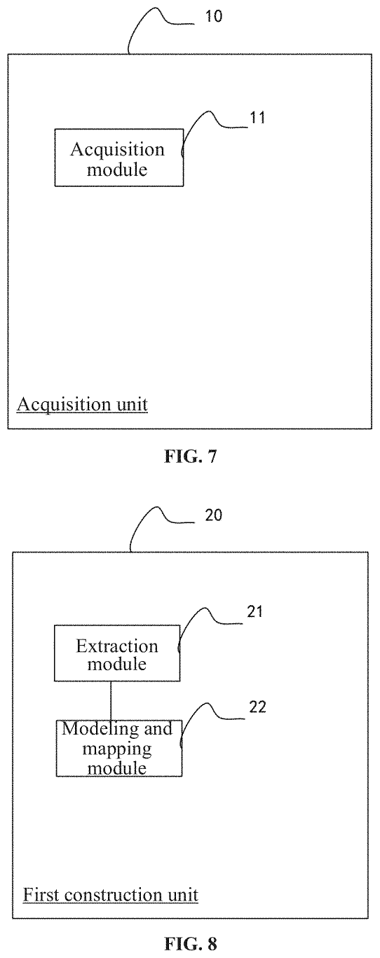

In an embodiment, as shown in FIG. 7, the acquisition unit 10 may include an acquisition module 11.

The acquisition module 11 is configured to acquire an arterial blood pressure, a venous blood pressure, an arterial compliance and a peripheral impedance of the cardio-aortic system in the complete cardiac cycle under the normal physiological state.

In the embodiment, these parameters such as the arterial blood pressure, the venous blood pressure, the arterial compliance and the peripheral impedance are acquired to facilitate the subsequent calculation to obtain the dynamic arterial blood flow volume.

In an embodiment, as shown in FIG. 8, the first construction unit 20 may include an extraction module 21, and a modeling and mapping module 22.

The extraction module 21 is configured to extract a medical image of the cardio-aortic system at a maximum end diastolic volume and a minimum end systolic volume of the left ventricle from morphological and motion imaging data of the cardio-aortic system in one complete cardiac cycle.

The modeling and mapping module 22 is configured to perform modeling and mapping according to the medical image of the cardio-aortic system to obtain the circulation dynamic loading function for the cyclic dynamic contraction of the left ventricle.

In the embodiment, the physiological pulsation function for the left ventricle of the heart is implemented through model mapping from the maximum end diastolic volume to the minimum end systolic volume of the left ventricle of the heart.

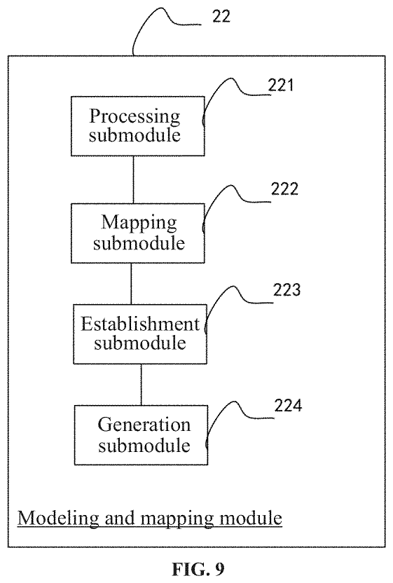

In an embodiment, as shown in FIG. 9, the modeling and mapping module 22 may include a processing submodule 221, a mapping submodule 222, an establishment submodule 223 and a generation submodule 224.

The processing submodule 221 is configured to construct a maximum end diastolic volume model and a minimum end systolic volume model respectively according to the medical image of the cardio-aortic system through image extracting, geometric modeling and finite element modeling.

The mapping submodule 222 is configured to construct a grid unit node mapping between the maximum end diastolic volume model and the minimum end systolic volume model, to determine a point-to-point corresponding relation between the grid unit nodes.

The establishment submodule 223 is configured to establish an LV volume change function V_{LV}(t) according to the morphological and motion imaging data of the cardio-aortic system in the complete cardiac cycle.

The generation submodule 224 is configured to obtain a motion displacement function WO on the basis of the mapping in combination with the LV volume change function V_{LV}(t), and take the motion displacement function Dᵢ(t) as the circulation dynamic loading function for the cyclic dynamic contraction of the left ventricle.

In the embodiment, the physiological pulsation function for the left ventricle of the heart is implemented through model mapping from the maximum end diastolic volume to the minimum end systolic volume of the left ventricle of the heart based on the medical image of the cardio-aortic system and the finite element method.

In an embodiment, as shown in FIG. 10, the second construction unit 30 may include a first construction module 31.

The first construction module 31 is configured to construct the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)(V_{LV}(t)−V_{LVmin})+E_{P}(V_{LV}(t)−V_{LVmax}), where P_{LV(t)} is the LV dynamic blood pressure, V_{LV}(t) is the LV dynamic volume, V_{LVmax} is the maximum end diastolic volume, V_{LVmin} is the minimum end systolic volume, E_{A}(t) is a time-varying elasticity coefficient for LV myocardial active contraction, and E_{P} is a passive elasticity coefficient.

In the embodiment, the simulation of aspects from pulsation of the heart to the dynamic blood pressure is implemented by constructing the dynamic relation function between the volume and the pressure of the left ventricle, which can effectively improve the level of accuracy of modeling and research on functions and injuries of the cardio-aortic system.

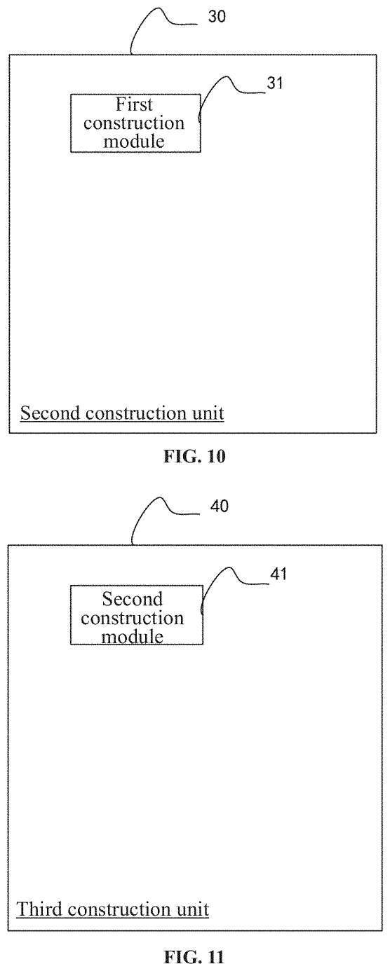

In an embodiment, as shown in FIG. 11, the third construction unit 40 may include a second construction module 41.

The second construction module 41 is configured to construct the dynamic arterial blood flow volume function according to the arterial blood pressure, the venous blood pressure, the arterial compliance and the peripheral impedance by employing

$$Q ( t ) = C \frac{dP ( t )}{d ι} + \frac{P ( t ) - {P}_{V}}{{R}_{P}} ,$$

where Q(t) is an arterial blood flow volume, P(t) is the arterial blood pressure, P_{V} is the venous blood pressure, C is the arterial compliance, and R_{P} is the peripheral impedance.

In the embodiment, the simulation of aspects from pulsation of the heart to the dynamic blood pressure is implemented by constructing the dynamic arterial blood flow volume function, which can effectively improve the level of accuracy of modeling and research on functions and injuries of the cardio-aortic system.

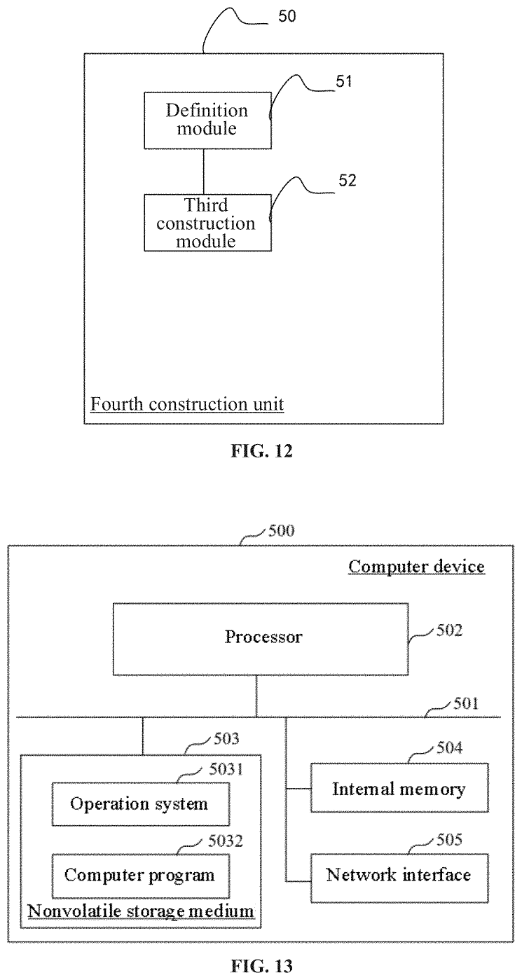

In an embodiment, as shown in FIG. 12, the fourth construction unit 50 may include a definition module 51 and a third construction module 52.

The definition module 51 is configured to define, based on the maximum end diastolic volume model and the minimum end systolic volume model, a region of the cardio-aortic system as an Euler fluid grid filled with two materials, with an LV wall and an aortic wall as dynamic limits, and the inside is automatically defined as a blood fluid, and the outside is automatically defined as an air fluid.

The third construction module 52 is configured to control a contact relation between the region-defined aortic valve and blood with 0-1 activated states of a preset function A(t), and reversely control a contact relation between the region-defined LV wall and the blood with 0-1 activated states of a preset function |A(t)−1|, through an ALE algorithm, thereby constructing the cyclic opening and closing activation function of the aortic valve.

In the embodiment, the contact relation between the region-defined aortic valve and the blood is controlled with the 0-1 activated states of the preset function A(t) (activated in a diastolic phase to avoid blood reflux of the aorta; and deactivated in a systolic phase to ensure blood pumping of the ventricle), and the contact relation between the region-defined LV wall and the blood is reversely controlled with the 0-1 activated states of the preset function |A(t)−1| (activated in the systolic phase to ensure blood pumping of the ventricle; and deactivated in the diastolic phase to ensure blood filling of the ventricle), to construct the cyclic opening and closing activation function of the aortic valve, and ensure that dynamic functional simulation of the constructed model is close to a physiological cyclic change of a real cardio-aortic system. The apparatus implements simulation on dynamic physiological characteristics of the cardio-aortic system in the complete cardiac cycle, and is used in the fields of life science, medicines, biomechanics, rehabilitation engineering and safety technologies to research the physiological characteristics, functional injuries, injury mechanisms and the like and an injury diagnosis and treatment of the cardio-aortic system under a virtual state.

It is to be noted that those skilled in the art may clearly understand that the specific implementation process of the apparatus for simulating the physiological dynamic blood flow and each unit may refer to the corresponding descriptions in the method embodiment, which will not be repeated herein for the sake of convenience and simplicity.

Referring to FIG. 13, FIG. 13 is a schematic block diagram of a computer device provided by an embodiment of the present disclosure. The computer device 500 may be a terminal, and may also be a server, and the terminal may be an electronic device having a communication function such as a smartphone, a tablet computer, a notebook computer, a desktop computer, a personal digital assistant and a wearable device. The server may be a standalone server, and may also be a server cluster composed of multiple servers.

Referring to FIG. 13, the computer device 500 includes a processor 502, a memory and a network interface 505 that are connected by a system bus 501. The memory may include a nonvolatile storage medium 503 and an internal memory 504.

The nonvolatile storage medium 503 may store an operation system 5031 and a computer program 5032. The computer program 5032 includes a program instruction. When executed, the program instruction may cause the processor 502 to execute the method for simulating a physiological dynamic blood flow.

The processor 502 is configured to provide computing and control capabilities to support the operation of the entire computer device 500.

The internal memory 504 provides an environment for the operation of the computer program 5032 in the nonvolatile storage medium 503. When executed by the processor 502, the computer program 5032 may cause the processor 502 to execute the method for simulating a physiological dynamic blood flow.

The network interface 505 is configured for network communication with other devices. Those skilled in the art may understand that the structure shown in FIG. 13 is only a block diagram on a part of the structure related to the solution of the application and does not constitute a limitation on the computer device 500 to which the solution of the application is applied. Specifically, the computer device 500 may include more or less components than those shown in the figures, or combine some components, or have different component arrangements.

The processor 502 is configured to run the computer program 5032 stored in the memory.

It should be understood that in the embodiment of the application, the processor 502 may be a Central Processing Unit (CPU); and the processor 502 may also be another general purpose processor, a Digital Signal Processor (DSP), an Application Specific Integrated Circuit (ASIC), a Field-Programmable Gate Array (FPGA) or another programmable logic device (PLD), a discrete gate, a transistor logic device, a discrete hardware component, etc. The general purpose processor may be a microprocessor, or the processor may also be any conventional processor, or the like.

Those of ordinary skill in the art may understand that, all or a part of the processes of the method in the preceding embodiments may be implemented by a computer program instructing relevant hardware. The computer program includes a program instruction. The computer program may be stored in a storage medium. The storage medium is a computer-readable storage medium. The program instruction is executed by at least one processor in the computer system, to implement the steps of the embodiments of the method.

Therefore, the present disclosure further provides a storage medium. The storage medium may be a computer-readable storage medium.

The storage medium may be various computer-readable storage media capable of storing a program code such as a U disk, a mobile hard disk, a Read-Only Memory (ROM), a magnetic disk or an optical disc.

Those of ordinary skill in the art may realize that, the units and the steps of the examples of the algorithms described in the embodiments of the present disclosure can be implemented with electronic hardware, computer software, or a combination thereof. In order to clearly describe the interchangeability between the hardware and the software, compositions and steps of each example have been generally described according to functions in the foregoing descriptions. Whether these functions are implemented by using hardware or software depends on the specific application of the technical solutions and design constraints. Those skilled in the art may use different methods to implement the described functions for each specific application, but such implementation should not be considered to be beyond the scope of the present disclosure.

In the several embodiments provided in the present disclosure, it should be understood that the disclosed apparatus and method may be implemented in other manners. For example, the described apparatus embodiment is merely exemplary. For example, the unit division is merely logical function division and may be other division in actual implementation. For example, multiple units or components may be combined or integrated into another system, or some features may be ignored or not performed.

The steps in the method of the embodiments of the present disclosure may be sequentially adjusted, merged, and deleted according to actual needs. The units in the apparatus of the embodiments of the present disclosure may be merged, divided and deleted according to actual needs. In addition, each functional unit in the embodiments of the present disclosure may be integrated into one processing unit, or each unit may exist alone physically, or two or more units are integrated into one unit.

If implemented in the form of a software functional unit and sold or used as a stand-alone product, the integrated unit may be stored in a storage medium. Based on such understanding, the technical solution of the present disclosure in essence or a part contributing to the prior art or all or a part of the technical solution may be embodied in the form of a software product. The computer software product is stored in a storage medium and includes multiple instructions for enabling a computer device (which may be a personal computer, a terminal, or a network device, etc.) to execute all or a part of steps of the method according to each embodiment of the present disclosure.

The above merely describes specific embodiments of the present disclosure, but the protection scope of the present disclosure is not limited thereto. Any person skilled in the art can easily conceive equivalent modifications or replacements within the technical scope of the present disclosure, and these modifications or replacements shall fall within the protection scope of the present disclosure. Therefore, the protection scope of the present disclosure should be subject to the protection scope of the claims.

What is claimed is:

1. A method for simulating a physiological dynamic blood flow, comprising the following steps:
acquiring related parameters of a cardio-aortic system in a complete cardiac cycle under a normal physiological state, and acquiring morphological and motion imaging data of the cardio-aortic system in the complete cardiac cycle;
constructing a circulation dynamic loading function for cyclic dynamic contraction of a left ventricle according to the morphological and motion imaging data of the cardio-aortic system in the complete cardiac cycle;
constructing a function between a left ventricular (LV) dynamic blood pressure and an LV dynamic volume according to the morphological and motion imaging data of the cardio-aortic system in the complete cardiac cycle;
constructing a dynamic arterial blood flow volume function according to the related parameters of the cardio-aortic system in the complete cardiac cycle under the normal physiological state;
constructing a cyclic opening and closing activation function of an aortic valve based on the morphological and motion imaging data of the cardio-aortic system in the complete cardiac cycle; and
constructing a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulating the physiological dynamic blood flow according to the physiological dynamic blood flow model.

2. The method for simulating a physiological dynamic blood flow according to claim 1, wherein the step of acquiring related parameters of a cardio-aortic system in a complete cardiac cycle under a normal physiological state comprises:
acquiring an arterial blood pressure, a venous blood pressure, an arterial compliance and a peripheral impedance of the cardio-aortic system in the complete cardiac cycle under the normal physiological state.

3. The method for simulating a physiological dynamic blood flow according to claim 2, wherein the step of constructing a circulation dynamic loading function for cyclic dynamic contraction of a left ventricle according to the morphological and motion imaging data of the cardio-aortic system in the complete cardiac cycle comprises:
extracting a medical image of the cardio-aortic system at a maximum end diastolic volume and a minimum end systolic volume of the left ventricle from morphological and motion imaging data of the cardio-aortic system in one complete cardiac cycle; and
performing modeling and mapping according to the medical image of the cardio-aortic system to obtain the circulation dynamic loading function for the cyclic dynamic contraction of the left ventricle.

4. The method for simulating a physiological dynamic blood flow according to claim 3, wherein the step of performing modeling and mapping according to the medical image of the cardio-aortic system to obtain the circulation dynamic loading function for the cyclic dynamic contraction of the left ventricle comprises:
constructing a maximum end diastolic volume model and a minimum end systolic volume model respectively according to the medical image of the cardio-aortic system through image extracting, geometric modeling and finite element modeling;
constructing a grid unit node mapping between the maximum end diastolic volume model and the minimum end systolic volume model to determine a point-to-point corresponding relation between the grid unit nodes;
establishing an LV volume change function V_{LV}(t) according to the morphological and motion imaging data of the cardio-aortic system in the complete cardiac cycle; and
obtaining a motion displacement function Dᵢ(t) on the basis of the mapping in combination with the LV volume change function V_{LV}(t), and taking the motion displacement function Dᵢ(t) as the circulation dynamic loading function for the cyclic dynamic contraction of the left ventricle.

5. The method for simulating a physiological dynamic blood flow according to claim 4, wherein the step of constructing a function between an LV dynamic blood pressure and an LV dynamic volume according to the morphological and motion imaging data of the cardio-aortic system in the complete cardiac cycle comprises:
constructing the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)(V_{LV}(t)−V_{LVmin})+E_{P}(V_{LV}(t)−V_{LVmax}), wherein P_{LV(t)} is the LV dynamic blood pressure, V_{LV}(t) is the LV dynamic volume, V_{LVmax} is the maximum end diastolic volume, V_{LVmin} is the minimum end systolic volume, E_{A}(t) is a time-varying elasticity coefficient for LV myocardial active contraction, and E_{P} is a passive elasticity coefficient.

6. The method for simulating a physiological dynamic blood flow according to claim 5, wherein the step of constructing a dynamic arterial blood flow volume function according to the related parameters of the cardio-aortic system in the complete cardiac cycle under the normal physiological state comprises:
constructing the dynamic arterial blood flow volume function according to the arterial blood pressure, the venous blood pressure, the arterial compliance and the peripheral impedance by using
$$Q ( t ) = C \frac{dP ( t )}{d ι} + \frac{P ( t ) - {P}_{V}}{{R}_{P}} ,$$
wherein Q(t) is an arterial blood flow volume, P(t) is the arterial blood pressure, P_{V} is the venous blood pressure, C is the arterial compliance, and Rp is the peripheral impedance.

7. The method for simulating a physiological dynamic blood flow according to claim 6, wherein the step of constructing a cyclic opening and closing activation function of an aortic valve based on the morphological and motion imaging data of the cardio-aortic system in the complete cardiac cycle comprises:
defining, based on the maximum end diastolic volume model and the minimum end systolic volume model, a region of the cardio-aortic system as an Euler fluid grid filled with two materials with an LV wall and an aortic wall as dynamic limits, wherein the inside is automatically defined as a blood fluid, and the outside is automatically defined as an air fluid; and
controlling a contact relation between the region-defined aortic valve and blood with 0-1 activated states of a preset function A(t), and reversely controlling a contact relation between the region-defined LV wall and the blood with 0-1 activated states of a preset function |A(t)−1|, through an arbitrary Lagrangian Eulerian (ALE) algorithm, thereby constructing the cyclic opening and closing activation function of the aortic valve.

8. An apparatus for simulating a physiological dynamic blood flow, comprising:
an acquisition unit, configured to acquire related parameters of a cardio-aortic system in a complete cardiac cycle under a normal physiological state, and morphological and motion imaging data of the cardio-aortic system in the complete cardiac cycle;
a first construction unit, configured to construct a circulation dynamic loading function for cyclic dynamic contraction of a left ventricle according to the morphological and motion imaging data of the cardio-aortic system in the complete cardiac cycle;
a second construction unit, configured to construct a function between a left ventricular (LV) dynamic blood pressure and an LV dynamic volume according to the morphological and motion imaging data of the cardio-aortic system in the complete cardiac cycle;
a third construction unit, configured to construct a dynamic arterial blood flow volume function according to the related parameters of the cardio-aortic system in the complete cardiac cycle under the normal physiological state;
a fourth construction unit, configured to construct a cyclic opening and closing activation function of an aortic valve based on the morphological and motion imaging data of the cardio-aortic system in the complete cardiac cycle; and
a model generation unit, configured to construct a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulate the physiological dynamic blood flow according to the physiological dynamic blood flow model.

9. The apparatus for simulating a physiological dynamic blood flow according to claim 8, wherein the acquisition unit comprises:
an acquisition module, configured to acquire an arterial blood pressure, a venous blood pressure, an arterial compliance and a peripheral impedance of the cardio-aortic system in the complete cardiac cycle under the normal physiological state.

10. The apparatus for simulating a physiological dynamic blood flow according to claim 9, wherein the first construction unit comprises:
an extraction module, configured to extract a medical image of the cardio-aortic system at a maximum end diastolic volume and a minimum end systolic volume of the left ventricle from morphological and motion imaging data of the cardio-aortic system in one complete cardiac cycle;
a modeling and mapping module, configured to perform modeling and mapping according to the medical image of the cardio-aortic system to obtain the circulation dynamic loading function for the cyclic dynamic contraction of the left ventricle.

11. The apparatus for simulating a physiological dynamic blood flow according to claim 10, wherein the modeling and mapping module comprises:
a processing submodule, configured to construct a maximum end diastolic volume model and a minimum end systolic volume model respectively according to the medical image of the cardio-aortic system through image extracting, geometric modeling and finite element modeling a mapping submodule, configured to construct a grid unit node mapping between the maximum end diastolic volume model and the minimum end systolic volume model, to determine a point-to-point corresponding relation between the grid unit nodes;
an establishment submodule, configured to establish an LV volume change function V_{LV}(t) according to the morphological and motion imaging data of the cardio-aortic system in the complete cardiac cycle; and
a generation submodule, configured to obtain a motion displacement function WO on the basis of the mapping in combination with the LV volume change function V_{LV}(t), and take the motion displacement function Dᵢ(t) as the circulation dynamic loading function for the cyclic dynamic contraction of the left ventricle.

12. The apparatus for simulating a physiological dynamic blood flow according to claim 11, wherein the second construction unit comprises:
a first construction module, configured to construct the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)(V_{LV}(t)−V_{LVmin})+E_{P}(V_{LV}(t)−V_{LVmax}), where P_{LV}(t) is the LV dynamic blood pressure, V_{LV}(t) is the LV dynamic volume, V_{LVmax} is the maximum end diastolic volume, V_{LVmin} is the minimum end systolic volume, E_{A}(t) is a time-varying elasticity coefficient for LV myocardial active contraction, and E_{P} is a passive elasticity coefficient.

13. A computer device, comprising a memory and a processor, wherein a computer program is stored on the memory, and when executed by the processor, the computer program implements the method for simulating a physiological dynamic blood flow comprises:
acquiring related parameters of a cardio-aortic system in a complete cardiac cycle under a normal physiological state, and acquiring morphological and motion imaging data of the cardio-aortic system in the complete cardiac cycle;
constructing a circulation dynamic loading function for cyclic dynamic contraction of a left ventricle according to the morphological and motion imaging data of the cardio-aortic system in the complete cardiac cycle;
constructing a function between a left ventricular (LV) dynamic blood pressure and an LV dynamic volume according to the morphological and motion imaging data of the cardio-aortic system in the complete cardiac cycle;
constructing a dynamic arterial blood flow volume function according to the related parameters of the cardio-aortic system in the complete cardiac cycle under the normal physiological state;
constructing a cyclic opening and closing activation function of an aortic valve based on the morphological and motion imaging data of the cardio-aortic system in the complete cardiac cycle; and
constructing a physiological dynamic blood flow model according to the circulation dynamic loading function, the function between the LV dynamic blood pressure and the LV dynamic volume, the dynamic arterial blood flow volume function and the cyclic opening and closing activation function of the aortic valve, and simulating the physiological dynamic blood flow according to the physiological dynamic blood flow model.

14. The computer device according to claim 13, wherein the step of acquiring related parameters of a cardio-aortic system in a complete cardiac cycle under a normal physiological state comprises:
acquiring an arterial blood pressure, a venous blood pressure, an arterial compliance and a peripheral impedance of the cardio-aortic system in the complete cardiac cycle under the normal physiological state.

15. The computer device according to claim 14, wherein the step of constructing a circulation dynamic loading function for cyclic dynamic contraction of a left ventricle according to the morphological and motion imaging data of the cardio-aortic system in the complete cardiac cycle comprises:
extracting a medical image of the cardio-aortic system at a maximum end diastolic volume and a minimum end systolic volume of the left ventricle from morphological and motion imaging data of the cardio-aortic system in one complete cardiac cycle; and
performing modeling and mapping according to the medical image of the cardio-aortic system to obtain the circulation dynamic loading function for the cyclic dynamic contraction of the left ventricle.

16. The computer device according to claim 15, wherein the step of performing modeling and mapping according to the medical image of the cardio-aortic system to obtain the circulation dynamic loading function for the cyclic dynamic contraction of the left ventricle comprises:
constructing a maximum end diastolic volume model and a minimum end systolic volume model respectively according to the medical image of the cardio-aortic system through image extracting, geometric modeling and finite element modeling;
constructing a grid unit node mapping between the maximum end diastolic volume model and the minimum end systolic volume model to determine a point-to-point corresponding relation between the grid unit nodes;
establishing an LV volume change function V_{LV}(t) according to the morphological and motion imaging data of the cardio-aortic system in the complete cardiac cycle; and
obtaining a motion displacement function Dᵢ(t) on the basis of the mapping in combination with the LV volume change function V_{LV}(t), and taking the motion displacement function Dᵢ(t) as the circulation dynamic loading function for the cyclic dynamic contraction of the left ventricle.

17. The computer device according to claim 16, wherein the step of constructing a function between an LV dynamic blood pressure and an LV dynamic volume according to the morphological and motion imaging data of the cardio-aortic system in the complete cardiac cycle comprises:
constructing the function between the LV dynamic blood pressure and the LV dynamic volume according to the LV volume change function V_{LV}(t), and the maximum end diastolic volume and the minimum end systolic volume of the left ventricle by employing P_{LV(t)}=E_{A}(t)(V_{LV}(t)−V_{LVmin})+E_{P}(V_{LV}(t)−V_{LVmax}), wherein P_{LV(t)} is the LV dynamic blood pressure, V_{LV}(t) is the LV dynamic volume, V_{LVmax} is the maximum end diastolic volume, V_{LVmin} is the minimum end systolic volume, E_{A}(t) is a time-varying elasticity coefficient for LV myocardial active contraction, and E_{P} is a passive elasticity coefficient.

18. The computer device according to claim 17, wherein the step of constructing a dynamic arterial blood flow volume function according to the related parameters of the cardio-aortic system in the complete cardiac cycle under the normal physiological state comprises:
constructing the dynamic arterial blood flow volume function according to the arterial blood pressure, the venous blood pressure, the arterial compliance and the peripheral impedance by using
$$Q ( t ) = C \frac{dP ( t )}{d ι} + \frac{P ( t ) - {P}_{V}}{{R}_{P}} ,$$
wherein Q(t) is an arterial blood flow volume, P(t) is the arterial blood pressure, P_{V} is the venous blood pressure, C is the arterial compliance, and R_{P} is the peripheral impedance.

19. The computer device according to claim 18, wherein the step of constructing a cyclic opening and closing activation function of an aortic valve based on the morphological and motion imaging data of the cardio-aortic system in the complete cardiac cycle comprises:
defining, based on the maximum end diastolic volume model and the minimum end systolic volume model, a region of the cardio-aortic system as an Euler fluid grid filled with two materials with an LV wall and an aortic wall as dynamic limits, wherein the inside is automatically defined as a blood fluid, and the outside is automatically defined as an air fluid; and
controlling a contact relation between the region-defined aortic valve and blood with 0-1 activated states of a preset function A(t), and reversely controlling a contact relation between the region-defined LV wall and the blood with 0-1 activated states of a preset function |A(t)−1|, through an arbitrary Lagrangian Eulerian (ALE) algorithm, thereby constructing the cyclic opening and closing activation function of the aortic valve.

20. A storage medium, storing a computer program, wherein when executed by a processor, the computer program implements the method for simulating a physiological dynamic blood flow according to any one of claim 1.
